(19) United States
(12) Patent Application Publication
(10) Pub. No.: US 2025/0114328 A1
(43) Pub. Date: Apr. 10, 2025

(54) VAPORIZABLE COMPOSITIONS OF 5-MeO-DMT AND THEIR USE

(71) Applicant: PAX LABS, INC., San Francisco, CA (US)

(72) Inventor: Richard RUCKER, San Francisco, CA (US)

(73) Assignee: PAX LABS, INC., San Francisco, CA (US)

(21) Appl. No.: 18/910,751

(22) Filed: Oct. 9, 2024

Related U.S. Application Data
(60) Provisional application No. 63/543,313, filed on Oct. 10, 2023.

(51) Int. Cl. A61K 31/4045; A24B 15/167; A61K 9/00; A61K 31/00; A61K 47/10; A61K 47/14

(52) U.S. Cl. CPC A61K 31/4045; A24B 15/167; A61K 9/0078; A61K 31/658; A61K 47/10; A61K 47/14

(57) ABSTRACT

The present application describes compositions comprising 5-MeO-DMT, one or more diluents, and optionally a cannabinoid compound for inhalation through vaporization. In certain embodiments, the compositions can be utilized as a treatment of trauma-related disorder or stressor-related disorder, such as post-traumatic stress disorder (PTSD), acute stress disorder (ASD), reactive attachment disorder (RAD), and disinhibited social engagement disorder (DSED). In particular embodiments, the composition comprises about 15-45% 5-MeO-DMT and 15-35% CBD dissolved in triethyl citrate or alpha-bisabolol, which has been observed herein to effectively remedy patients' anxiety and stress.

### CROSS REFERENCE TO RELATED APPLICATION

This application claims priority to U.S. Provisional Application No. 63/543,313, filed on Oct. 10, 2023, the disclosure of which is incorporated herein by reference in its entirety.

### BACKGROUND OF THE INVENTION

Treating psychological disorders with the use of cannabis has become a primary application of the cannabis industry. When consumed, certain cannabinoid compositions have a unique ability to reduce symptoms of anxiety and depression, which are commonly seen in individuals with trauma-related or stressor-related disorders. For example, certain cannabinoids can interact with cannabinoid receptors (the two main types are CB1 and CB2) in the brain and nervous system, which can produce a sense of relaxation and euphoria. Such cannabinoid compositions generally comprise a combination of active compounds at varying concentration levels including but not limited to cannabidiol (CBD), tetrahydrocannabinol (THC), cannabichromene (CBC), cannabinol (CBN), cannabidvarin (CBDV), and cannabigerol (CBG).

Similarly, 5-methoxy-N, N-dimethyltryptamine (5-MeO-DMT), a psychoactive substance that belongs to the tryptamine class of compounds, has gained attention in recent years for its potential therapeutic uses in the treatment of depression, anxiety, and addiction. There have been some studies and case reports that suggest 5-MeO-DMT may be helpful in reducing symptoms of trauma-related or stressor-related disorders, such as post-traumatic stress disorder (PTSD).

Cannabinoid compositions show promise for treatment of trauma-related or stressor-related disorders, through a variety of routes including inhalation, oral ingestion, topical application (e.g., in the form of creams, balms, or lotions) and sublingual administration (e.g., placed under the tongue and absorbed through the mucous membranes). Among all these different routes, inhalation is widely considered the most effective because it allows for rapid onset, which is crucial for individuals experiencing acute symptoms. For example, inhalation typically has an onset time for effects of the cannabinoid compositions in the range of just a few minutes, oral ingestion typically provides onset times in the range of 15 minutes or more, often not providing peak effect until 60 minutes after ingestion.

Both cannabinoid compositions and 5-MeO-DMT are generally in solid or semisolid state at room temperature (68° F./20°° C. to 78° F./26°° C.), and thus they need to be vaporized for patients to inhale. Vaporization involves heating the substance to a temperature that is high enough to convert its active compounds into gaseous vapor, but not too high to cause combustion of the substance. Furthermore, the vaporization efficiency and the range of viscosity could be significantly improved by dissolving the substance in certain diluents. The ratio between a substance and diluent is crucial for effective and efficient vaporization and viscosity control. Viscosity is the measurement of an internal resistance to flow. Viscosity arises from the momentum transfer between gas molecules as they collide with each other, which reflects the internal friction between layers of fluid as they move past one another.

There remains a need for improved compositions for vaporization of 5-MeO-DMT cannabinoid compounds. Such compositions can provide, among other things, improved treatments for psychological disorders.

### SUMMARY OF THE INVENTION

There exists a need to optimize efficiency of vaporization of compositions containing 5-MeO-DMT. The present application describes compositions of 5-MeO-DMT in one or more diluents, with or without the addition of cannabinoids.

In one embodiment, the present application describes compositions of 5-MeO-DMT and one or more diluents for use in a vaporization device. In certain embodiments, the composition comprises a viscosity of between about 400 cP and about 150,000 cp. In particular examples, the composition comprises a viscosity of between about 600 cP and about 100,000 cp; between about 1,000 cP and about 50,000 cp; or between about 1,500 cP and about 20,000 cp.

In certain embodiments, the weight percentage of 5-MeO-DMT in the composition is ranging from about 1 wt % to about 99 wt %. In particular examples, the weight percentage of 5-MeO-DMT is ranging from about 5 wt % to about 90 wt %, from about 10 wt % to about 70 wt %, from about 15 wt % to about 50 wt %, or from about 15 wt % to about 25 wt %.

In certain embodiments, one or more diluents are selected from the group consisting of triethyl citrate, propylene glycol, vegetable glycerin, polyethylene glycol, terpenoids, and cannabinoid distillates. In particular examples, terpenoids comprise one or more terpene alcohols, such as a-bisabolol and/or phytol. In other particular examples, the diluent is a cannabinoid distillate, such as a distillate comprising primarily CBD and/or THC, in some cases with other cannabinoids present, such as CBG, CBC, and/or CBN. In certain examples, the distillate comprises about 10-99% cannabinoids, such as about 5-90%, about 10-80%, about 10-70 %, about 15-60%, about 20-50%, about 25-50%, about 30-45%, about 35-40%, or about 40% cannabinoids.

In certain embodiments, the diluent achieves a viscosity range suitable for vaporization, resulting in less leaking and clogging in a vape system, more consistent dosing in a vape system, and/or reduced thermal degradation byproducts in a vape system.

In certain embodiments, the composition further comprises one or more cannabinoid compounds. In particular examples, the one or more cannabinoid compounds comprise one or more cannabinoids selected from the group consisting of delta-9-tetrahydrocannabinol (THC), delta-8-tetrahydrocannabinol (d8-THC), cannabichromene (CBC), cannabidiol (CBD), cannabichromene (CBC), cannabinol (CBN), cannabidvarin (CBDV), and cannabigerol (CBG). In other examples, one or more cannabinoids are in viscous or semisolid state at a temperature between about 68° F./20° C. and about 78° F./26° C. In other examples, one or more cannabinoids are in solid state at a temperature between about 68° F./20° C. and about 78° F./26° C.

In certain examples, 5-MeO-DMT is present at a concentration of at least about 20%, at least about 30%, at least about 40%, at least about 50%, at least about 60%, at least about 70%, at least about 75%, at least about 80%, at least about 85%, or at least about 90%.

In particular examples, the composition comprises 5-MeO-DMT and a diluent, wherein the diluent is a distillate comprising 30-50% cannabinoid, and 5-MeO-DMT is present at a concentration of 50-80%. In certain examples, the cannabinoid is CBD or THC. In other embodiments, cannabinoid is present in the distillate at a concentration of about 35%- 45% or at about 40%. In further embodiments, 5-MeO-DMT is present at a concentration at about 55%-75%, about 55%- 65%, about 60%, or about 75%.

In other examples, the ratio of 5-MeO-DMT to CBD is between 10:1 and 1:10. In particular examples, the ratio of 5-MeO-DMT to CBD is between 5:1 and 1:5; between 4:1 and 1:4; between 3:1 and 1:3; between 2:1 and 1:2; or between 1.5:1 and 1:1.5.

In other embodiments, the present application describes a method for treating a psychological disorder, the method comprising administering an effective amount of a composition described herein to a patient in need thereof using a vape system. In certain examples, the psychological disorder is a trauma-related disorder or stressor-related disorder. In particular examples, the trauma-related disorder or stressor-related disorder is selected from the group consisting of post-traumatic stress disorder (PTSD), acute stress disorder (ASD), reactive attachment disorder (RAD), and disinhibited social engagement disorder (DSED).

In certain examples, the composition is administered to a patient suffering from PTSD, wherein the patient experiences reduced symptoms of PTSD and/or experiences improved quality of life after administration of the composition.

In other embodiments, the composition is administered in combination with a second therapeutic intervention for treating the psychological disorder. In particular examples, the second therapeutic intervention is selected from the group consisting of counseling, psychotherapy, and medication.

### DETAILED DESCRIPTION OF THE INVENTION

It is to be understood that this disclosure is not limited to particular embodiments, which can, of course, vary. It is also to be understood that the terminology used herein is for the purpose of describing particular embodiments only and is not intended to be limiting. All patents and publications referred to herein are incorporated by reference. Unless otherwise defined, all technical and scientific terms used herein have the same meaning as commonly understood to one of ordinary skill in the art to which this invention pertains. Otherwise, certain terms used herein have the meanings as set forth in the specification.

As used in this specification and the appended claims, terms in the singular and the singular forms “a,” “an,” and “the,” for example, include plural referents unless the content clearly dictates otherwise. Thus, for example, reference to “weight,” “the weight” or “a weight” also includes a plurality of weights. Additionally, as used herein, the term “comprises” is intended to indicate a non-exhaustive list of components or steps, thus indicating that the given composition or method includes the listed components or steps and may also include additional components or steps not specifically listed. As an example, formulation “comprising THC” may also include additional components, such as CBD, terpenes, buffering components, etc. The term “comprising” is also intended to encompass embodiments “consisting essentially of” and “consisting of” the listed components or steps. Similarly, the term “consisting essentially of” is also intended to encompass embodiments “consisting of” the listed components or steps.

Numeric ranges recited within the specification and claims are inclusive of the numbers defining the range (the end point numbers) and also are intended to include each integer or any non-integer fraction within the defined range. Further, as used herein, the term “about” refers to a number that differs from the given number by 10% or less. In other embodiments, the term “about” indicates that the number differs from the given number by less than 9%, 8%, 7%, 6%, 5%, 4%, 3%, 2%, or 1%.

The present inventors have developed novel compositions of 5-MeO-DMT alone or 5-MeO-DMT with cannabinoid that are suitable for vaporization. In certain embodiments, the compositions can provide for treatment of psychological disorders when consumed, such as by inhalation of the vaporized composition. The formulations and compositions described herein can include one or more cannabinoids including but not limited to tetrahydrocannabinol (THC), cannabichromene (CBC), cannabidiol (CBD), cannabinol (CBN), cannabidvarin (CBDV), and cannabigerol (CBG). In certain embodiments, the formulation comprises tetrahydrocannabinol (THC), such as delta-9-THC or delta-8-THC. In particular embodiments, the formulation comprises delta-9-THC. In other embodiments, the composition comprises cannabidiol (CBD). In other embodiments, the composition comprises both THC and CBD. In further embodiments, the composition comprises one or more minor cannabinoids, such as CBC, CBG or CBN, optionally in combination with THC and/or CBD.

In further embodiments, the composition comprises one or more terpenoids, such as terpene alcohols. In particular examples, the presence of alpha bisabolol is dominant in concentration to the other terpene alcohols in the composition.

In particular embodiments, the composition comprises both 5-MeO-DMT and CBD in a ratio between about 10:1 and 1:10, such as a ratio of about 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10.

In particular examples, the composition comprises about 19-20% of 5-MeODMT in solutions of alpha-bisabolol as the diluent. In other particular examples, the composition comprises about 40-45%, such as about 44%, 5-MeODMT in solutions of alpha-bisabolol as the diluent. In particular examples, the viscosity of the composition is approximately 400-500 cP (in the case of the low wt % of 5-MeODMT) up to 100,000-150,000 cP (in the case of the high wt % of 5-MeODMT).

Determination of treatment of trauma-related or stressor-related disorders through inhalation of the compositions described herein can be by any suitable means. Methods for making such determinations are well known to persons of ordinary skill in the art, and a person of ordinary skill would be able to select appropriate methods.

Methods of determining impact of a composition/formulation on trauma-related or stressor-related disorders can include self-reporting of effects of the 5-MeO-DMT and cannabinoid compositions, such as feelings of relaxation or feelings of stress or anxiety felt by the subject. Self-reporting can include completion of surveys directed toward assessment of psychometric measures of stress and anxiety.

### EXAMPLES

The invention will now be described in connection with the following, non-limiting examples.

Example 1: 5-MeO-DMT with the addition of cannabidiol (CBD) for vaporization. A novel composition for vaporization in a vape system comprises 5-MeO-DMT combined with CBD. 5-MeO-DMT is in freebase powdered form. The CBD may contain minor amounts of other cannabinoids, such as d9-THC at less than 0.2 wt % (e.g., less than 0.0976 wt %). The tested effective ratio of the weight percentage between 5-MeO-DMT and CBD are: 22.7 wt % of 5-MeODMT with 30.6 wt % of CBD; 22.6 wt % of 5-MeODMT with 30.7 wt % of CBD; and 41.0 wt % of 5-MeODMT with 23.4 wt % of CBD. The compositions will treat psychological disorders when consumed by inhalation of the vaporized composition.

Example 2: 5-MeO-DMT dissolved in triethyl citrate. A composition for vaporization in a vape system comprises 5-MeODMT in solutions of triethyl citrate as the diluent. The tested effective weight percentage of 5-MeO-DMT suitable for vaporization are 35.3 wt %, 42.1 wt %, 46.9 wt %, and 68.8 wt %. The composition resulted in less leaking and clogging, more consistent dosing, and reduced thermal degradation byproducts in a vape system. The compositions will treat psychological disorders when consumed by inhalation of the vaporized composition.

Example 3: 5-MeO-DMT dissolved in alpha-bisabolol. A composition for vaporization in a vape system comprises 5-MeODMT in solutions of alpha-bisabolol as the diluent. The tested effective weight percentage of 5-MeO-DMT suitable for vaporization is 32.7 wt %. The composition resulted in less leaking and clogging, more consistent dosing, and reduced thermal degradation byproducts in a vape system. The compositions will treat psychological disorders when consumed by inhalation of the vaporized composition.

1. A composition for vaporization in a vape system, wherein said composition comprises 5-methoxy-N, N-dimethyltryptamine (5-MeO-DMT) and one or more diluents.

2. The composition of claim 1, wherein the composition comprises a viscosity of between about 1,500 cP and about 20,000 cP.

3. The composition of claim 1, wherein the weight percentage of 5-MeO-DMT is ranging from 30 wt % to 70 wt %.

4. The composition of claim 1, wherein the one or more diluents are selected from the group consisting of triethyl citrate, propylene glycol, vegetable glycerin, polyethylene glycol, and terpenoids.

5. The composition of claim 4, wherein the terpenoids comprise α-bisabolol and/or phytol.

6. The composition of claim 1, wherein the diluent achieves a viscosity range suitable for vaporization, resulting in less leaking and clogging, more consistent dosing, and reduced thermal degradation byproducts in a vape system.

7. A composition for vaporization in a vape system, wherein said composition comprises 5-methoxy-N, N-dimethyltryptamine (5-MeO-DMT), one or more cannabinoid compounds, and one or more diluents.

8. The composition of claim 7, wherein the composition comprises a viscosity of between about 1,500 cP and about 20,000 cP.

9. The composition of claims 7, wherein the weight percentage of 5-MeO-DMT is ranging from 20 wt % to 45 wt %.

10. The composition of claims 7, wherein the one or more cannabinoid compounds comprise one or more cannabinoids selected from the group consisting of delta-9-tetrahydrocannabinol (THC), cannabichromene (CBC), cannabidiol (CBD), cannabichromene (CBC), cannabinol (CBN), cannabidvarin (CBDV), and cannabigerol (CBG).

11. The composition of claim 10, wherein the cannabinoids are selected from the group consisting of CBD, CBC, CBN, CBDV and CBG, and wherein the cannabinoids are in a viscous, semisolid, or solid state at a temperature between 68° F. and 78° F.

12. (canceled)

13. The composition of claim 7, wherein the one or more diluents are selected from the group consisting of triethyl citrate, propylene glycol, vegetable glycerin, polyethylene glycol, and terpenoids.

14. The composition of claim 7, wherein the one or more diluents comprise terpenoids selected from α-bisabolol and/or phytol.

15. The composition of claims 7, wherein 5-MeO-DMT is present at a concentration of at least about 20%.

16. The composition of claims 7, wherein the ratio of 5-MeO-DMT to CBD is between 1.5:1 and 1:1.5.

17. The composition of claim 7, wherein the diluent achieves a viscosity range suitable for vaporization, resulting in less leaking and clogging, more consistent dosing, and reduced thermal degradation byproducts in a vape system.

18. A method for treating a psychological disorder, the method comprises administering an effective amount of the composition of claim 7 to a patient in need thereof using a vape system.

19. The method of claim 18, wherein the psychological disorder is a trauma-related disorder or stressor-related disorder, which is selected from the group consisting of post-traumatic stress disorder (PTSD), acute stress disorder (ASD), reactive attachment disorder (RAD), and disinhibited social engagement disorder (DSED).

20. The method of claim 19, wherein the trauma-related disorder or stressor-related disorder is (PTSD).

21. The method of claim 18, wherein the composition is administered in combination with a second therapeutic intervention for treating PTSD, which is selected from the group consisting of counseling, psychotherapy, and medication.

22. (canceled)
